(19) United States
(12) Patent Application Publication
(10) Pub. No.: US 2026/0224111 A1
(43) Pub. Date: Aug. 6, 2026

(54) SENSOR STRIPS FOR INTRINSIC NEAR INFRARED SPECTROSCOPY IMAGING

(71) Applicants: THE TRUSTEES OF DARTMOUTH COLLEGE, Hanover, NH (US); Wisconsin Alumni Research Foundation, Madison, WI (US)

(72) Inventors: Shudong JIANG, Hanover, NH (US); Brian W. POGUE, Madison, WI (US); Keith D. PAULSEN, Hanover, NH (US); Jinchao FENG, Hanover, NH (US); Jeffrey D. RENK, Hanover, NH (US); Xu CAO, Hanover, NH (US); Mingwei ZHOU, Hanover, NH (US); Mengyang ZHAO, Hanover, NH (US)

(21) Appl. No.: 19/152,334

(22) PCT Filed: Jan. 31, 2024
(86) PCT No.: PCT/US2024/013701

§ 371 Date: Jul. 30, 2025

Related U.S. Application Data
(60) Provisional application No. 63/442,418, filed on Jan. 31, 2023.

(51) Int. Cl. A61B 5/00

(52) U.S. Cl. CPC A61B 5/0075; A61B 5/0035; A61B 5/0091

(57) ABSTRACT

A near-infrared spectroscopy tomographic (NIRST) imaging strip has photodiodes for receiving light from diffuse media, the photodiodes and optical fiber probes in a line along a center of a flexible printed circuit; to deliver light from the optical fibers to the diffuse media. A method of performing imaging of diffuse media requires providing NIRST imaging strips each with a row of photodiodes and a slot for at least one optical fiber probe on a flexible circuit board; providing near-infrared light to the diffuse media to the optical fiber probes of each NIRST imaging strip receiving light directly from the diffuse media into photodiodes of the NIRST imaging strips; amplifying signals from the photodiodes; and recovering optical parameters of a voxel-based model of the diffuse media from the amplified signals from the plurality of photodiodes.

### PRIORITY CLAIM

This application claims priority to U.S. Provisional Patent Application 63/442,418 filed Jan. 31, 2023. The entire contents of the aforementioned application are incorporated herein by reference.

### BACKGROUND

Near infrared spectroscopy (NIRS) is an imaging technique that projects two or more wavelengths of infrared light into tissue at multiple injection points on the tissue and observes spectral changes in light returned from the tissue at multiple return, or detector, points on the tissue. The spectral changes at each return point are analyzed and reconstructed to form an image of the tissue.

NIRS contrast arises from the presence of blood, water and lipids as well as cellular structures that cause scattering and absorption at different wavelengths. NIRS provides a way to follow vascular density and functionality along with increased cellularity which are two of the predominant transformations associated with malignancy. For example, hemoglobin in blood serves to absorb near infrared (NIR) light at wavelengths dependent on hemoglobin oxygenation.

Non-invasive functional imaging stand-alone systems exist using NIRS. These image hemoglobin and other chromophores in diffuse media such as mammalian breast, brain, skin or other tissue. Hemoglobin levels are of particular interest because they are typically higher in tumors because rapidly dividing and growing tumor tissues require a good blood supply and secrete substances that encourage blood vessel growth. These tumors induce angiogenic hypervascularity and increased vessel permeability with up-regulation of several proendothelial cell growth factors like human epidermal growth factor receptor 2 (HER2) and/or Vascular endothelial growth factor (VEGF). Lower oxygen saturation and lipid levels often occur due to increased metabolic demands and lower adipose volume, respectively, in tumor tissue. Higher water content from edema can also be present in malignancies relative to normal tissues.

NIRS tomographic (NIRST) imaging devices have been used for breast cancer detection and therapeutic monitoring. While dynamic contrast-enhanced magnetic resonance imaging (DCE MRI) is recognized as the most sensitive examination for breast cancer detection, it has a substantial false positive rate and the gadolinium (Gd) contrast agents used in DCE-MRI appear to have safety risks. NIRS has been combined with non-contrast MRI and results showed when NIRS combined with non-contrast MRI achieved sensitivity, specificity, accuracy and area under the curve (AUC) of 94%, 100%, 96%, and 0.95, respectively for breast cancer detection.

These results are similar to those obtained with NIRST combined with DCE-MRI. NIRST alone has been used for monitoring and predicting the locally advanced breast cancer response to neoadjuvant chemotherapy (NAC). The clinical data from 35 patients undergoing NAC shows the change in total hemoglobin (ΔHbT %) by the end of chemotherapy cycle 1 versus pretreatment can predict pathological and clinical outcomes of response to NAC using residual cancer burden (RCB) class and score. RCB-0 can be differentiated from other classes accurately, (p<0.001). Corresponding Area Under the Curve (AUC) values for these comparisons were 0.97 and 0.94, and accuracy values were 0.90 and 0.83, respectively.

The long fiber bundles used by most NIRST systems to transport light to and from the breast from and to light sources and detectors on a system cart are bulky, difficult to use clinically, and challenging to position on the breast. Indeed, a clinical study showed that ~30% of exams resulted in low optical data sensitivity to the ROI (<1%) not because of poor fiber contact, small tumor size or large breast volume, but because of poor fiber placement and/or breast coverage.

### SUMMARY

A near-infrared spectroscopy tomographic (NIRST) imaging strip has photodiodes for receiving light from diffuse media, and optical fiber probes to deliver light from the lasers to the diffuse media. The photodiodes and fiber probes are in a line along a center of a flexible printed circuit.

A method of performing imaging of diffuse media requires providing NIRST imaging strips each with a row of photodiodes and a slot for at least one fiber probe on a flexible circuit board; providing near-infrared light to the diffuse media and receiving light directly from the diffuse media into photodiodes of the NIRST imaging strips; amplifying signals from the photodiodes; and recovering optical parameters of a voxel-based model of the diffuse media based on the tomographic reconstruction of the amplified signals from the plurality of photodiodes.

### BRIEF DESCRIPTION OF THE DRAWINGS

FIG. 1 is a block diagram of a system incorporating our sensor strips for performing NIRS imaging of diffuse media, including biological tissues such as the breast, in an embodiment.

FIG. 2 is a schematic layout diagram of a single sensor strip flexible printed circuit board layer showing photodiodes and optical fiber probe slots.

FIG. 3 is a layout diagram of optical fibers and optical fiber probes that may be attached to the flexible printed circuit board layer of FIG. 2 to form the sensor strip.

FIG. 4 is schematic layout diagram of a single sensor strip flexible printed circuit board layer.

FIG. 5 is a schematic layout diagram showing eight sensor strips in star configuration such as may be used for imaging the human breast.

FIG. 6 is a flowchart showing how the sensor strips are operated to image diffuse media such as a human breast.

FIGS. 7A-7F illustrate results obtained by experiment with the sensor strips on a breast phantom.

FIG. 7A illustrates a breast phantom,

FIG. 7B is a surface scan of strip-mounted fiber probes and photodiodes on the breast phantom,

FIG. 7C illustrates fiber probe head and photodiode positions on the breast phantom as entered as constraints in an infrared imaging software,

FIG. 7D illustrates an inclusion representing a tumor in the phantom as imaged with MRI,

FIG. 7E, and 7F illustrates an inclusion representing a tumor in the breast phantom, as imaged using the sensor strips on the breast phantom coupled to an infrared imaging system.

FIGS. 8A-H illustrate results obtained by imaging breast phantoms each with a spherical inclusion. The diameter of the inclusions are 10 mm, 15 mm, 20 mm and 25 mm, respectively. MRI and NIRST imaging were taken simultaneously.

FIGS. 8A, 8B, 8C and 8D illustrate inclusions representing a tumor in a phantom as imaged with MRI. The diameters of the inclusions are 10, 15, 20, and 25 millimeters, respectively.

FIGS. 8E, 8F, 8G, and 8H illustrate the corresponding NIRST total hemoglobin images as that in FIGS. 8A, 8B, 8C and 8D, respectively, using the sensor strips on the breast phantom coupled to an infrared imaging system combined with MRI extracted structure information according to the method of FIG. 9.

FIG. 9 illustrates use of the sensor strips and near-infrared imaging in an MRI system so that NIRST and MRI images can be captured simultaneously without movement of the patient between the NIRST and MRI image sets.

FIG. 10 is a flowchart illustrating a method of using the sensor strips herein described for performing breast imaging, the strips may also be used to image other parts of human and animal anatomy.

### DETAILED DESCRIPTION OF THE EMBODIMENTS

To overcome difficulties in handling fiber bundles common with existing NIRST systems in clinical practice, we provide flexible, MRI compatible, sensing strips that apply source fibers and electrooptical detectors directly to the breast, covering its entire volume, including the axilla region.

The sensing strips can be used with most NIRS diagnosis or imaging systems seamlessly and can significantly improve the diagnostic power of these commercialized systems. Indeed, the MRI compatibility of the strips may result in a new breast screening tool for breast cancer screen without need for MR contrast agent; the sensitivity of NIRS to hemoglobin concentration and hemoglobin oxygenation may serve to annotate MRI images to enable distinguishing malignant from nonmalignant lesions better than MR contrast agents can. As gadolinium-based MR contrast agents are recognized as being somewhat toxic, reduced or eliminated need for MR contrast agent can be advantageous particularly when imaging patients with impaired kidney function.

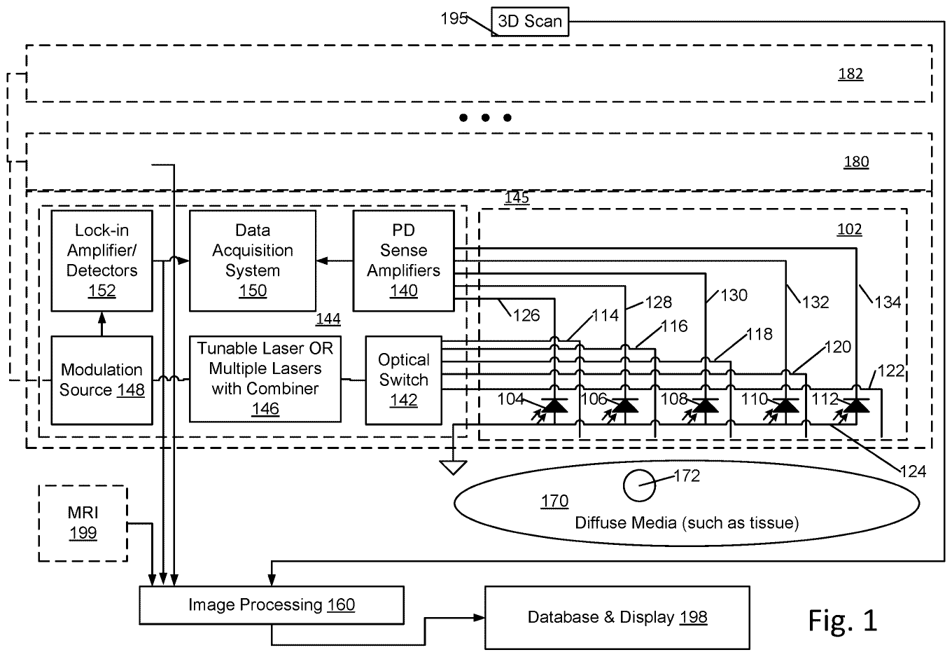

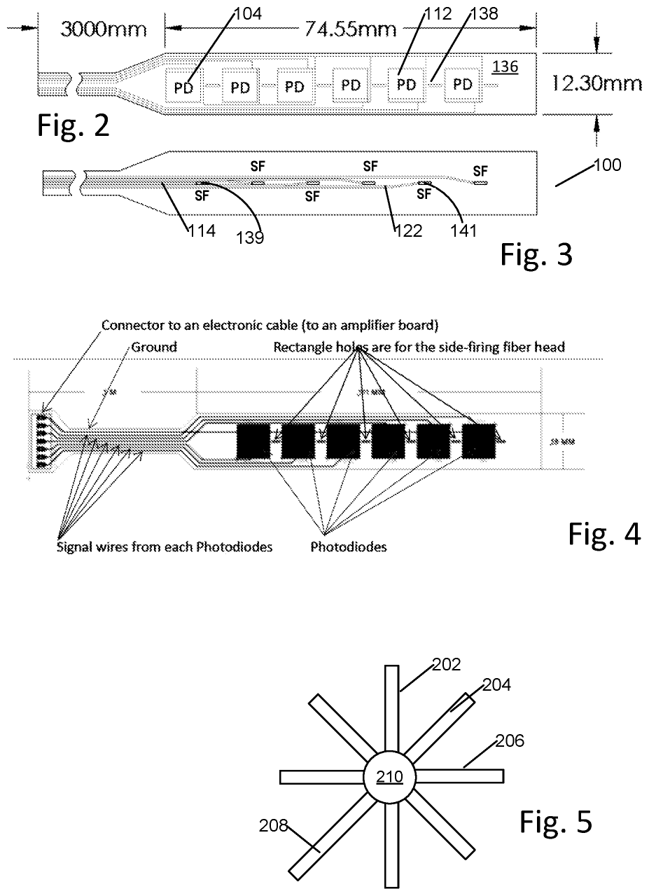

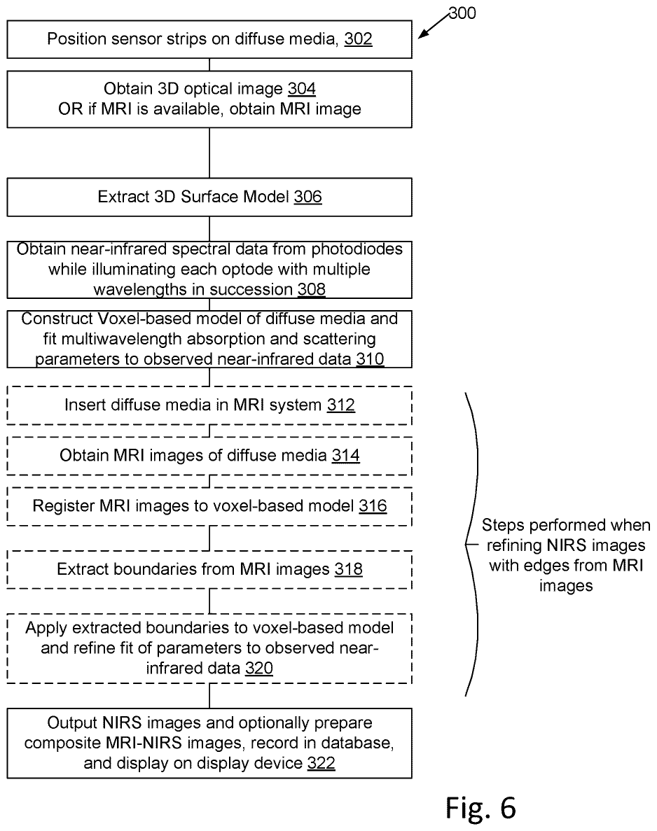

Our sensing strips 102 (FIG. 1) each have multiple photodiodes, in a particular embodiment five photodiodes 104, 106, 108, 110, 112. In alternative embodiments, as illustrated in FIG. 2, there are six photodiodes, and in other embodiments there may be two, three, four or more than six photodiodes. Each photodiode is tied to a common ground 124 and has a photocurrent signal brought out through separate conductive traces 126, 128, 130, 132, 134 on a flexible printed circuit 136 (FIG. 2) that forms a base for the strip. Flexible printed circuit 136 has slots 138 for insertion of right-angled or “side firing” (SF) fiber probes. As shown in FIG. 2, the photodiodes are evenly spaced and attached in line along a center of a 12.3 millimeter (mm) wide and 74.5 mm long portion of the strip. The flexible printed circuit of the flexible printed circuit 136 tapers to a cable portion having width about 5 mm and length about three meters; the cable portion terminates in a connector as shown in FIG. 4. Above the flexible printed circuit 136, as shown in FIGS. 1 and 3, is a flat strip formed of parallel optical fibers 114, 116, 118, 120, 122 bonded to a second substrate. Each of a first end of the optical fibers 114-122 terminates in an SF optical fiber probe 139,141 positioned in a slot 138 of flexible printed circuit 136.

In use, in each channel 145, the connector of the strip is coupled to photodiode signal amplifiers 140 (FIG. 1) of strip-associated electronics 144, thus coupling a signal amplifier of sense amplifiers 140 through conductive traces 126-134 to each photodiode of photodiodes 104-112. Similarly, a second end of each optical fiber 114-122 terminates in an optical switch 142 of strip-associated electronics 144. The optical switch 142 serves to selectively couple a fiber of fibers 114-122 to receive light from a selectable wavelength light source 146 that may be a tunable laser or may include multiple single-wavelength lasers with optical combiners. Selectable wavelength light source 146 is modulated with a waveform generated by a modulation source 148. Signal amplifiers 140 provide pre-filtered and amplified photodiode sensing information to a data acquisition system 150, such as a multichannel analog to digital converter, and thence to lock-in amplifiers 152 that serve to exclude noise from such sources as room light and in turn provides noise-filtered photodiode sense information to an image processing system 160.

The optical fibers 114-122 are configured to deliver light to diffuse media 170, which may in some instances be human or animal tissue, and in a particular embodiment is a human breast. Diffuse media 170 may have inclusions 172 such as a tumor. Photodiodes 104-112 are configured to receive light directly from diffuse media 170; for example, in an embodiment the photodiodes are disposed to directly contact the diffuse media without any intervening optical fibers although there may be a transparent window between the diffuse media and the photodiodes. The received light has been scattered and attenuated by the diffuse media 170 and inclusions 172.

While a single strip may be used for some purposes, in many embodiments multiple strips are used. There may be more than one, in some embodiments six, in others eight as illustrated in FIG. 5, channels 145, 180, 182 of sensor strip 102, 202, 204, 206, 208 deployed in star configuration over diffuse media 170 and coupled to image processor 160. In some embodiments all strips are the same length, in other embodiments a few strips, such as strip 208, may be longer with an additional photodiode, optical fiber, and optical fiber probe to better permit imaging of lymph nodes of the axilla as well as the breast itself. In a breast-imaging embodiment, the sensor strips may be joined together with a silicone nipple cup 210 with the sensor strips in star configuration. The lengthy cable portions are not shown in FIG. 5 for simplicity.

In some embodiments, each sensor strip 102, 202, 204, 205, 208 has an optically patterned coating for easy recognition by a three-dimensional (3-D) optical imaging system. In embodiments using these patterned strips, the 3D surface scanner 195 (FIG. 1) are provided to capture a stereo pair of images of sensor strips positioned on a subject or phantom, the image processing system 160 being configured to extract a 3-D surface model of the sensor strips as positioned on the diffuse media before or during near-infrared imaging of the diffuse media.

In operation according to method 300, the sensor strips are coupled to the associated electronics, then positioned 302 on the diffuse media, which in some cases is a human breast. The strips may be glued or taped to the diffuse media to retain them in position during imaging. In some embodiments, the sensor strips may be marked with a surface pattern to improve imaging results with 3D cameras or a 3D surface scanner. A 3D optical image is then obtained 304 using a 3D surface scanner 195. A 3D surface model is extracted 306 from the 3D optical image and used while constructing a three-dimensional voxel-based model 310 of the diffuse media. In embodiments where an MRI image is available, a three-dimensional voxel-based model 310 of the diffuse media may be extracted from the MRI image instead of a 3D optical image and the 3D surface scanner 195 may be omitted from the system.

Near-infrared spectral data is obtained 308 by repeatedly driving the optical fiber probes using the selectable wavelength light sources 146 in predetermined patterns with light of predetermined and varied wavelengths while recording received light intensity data at each of photodiodes 104-112. Optical parameters such as absorbance and scattering parameters at each wavelength and voxel are then reconstructed by the recorded light intensity data. This provides a raw three-dimensional near-infrared image of the diffuse media. Since optical parameters are obtained at multiple wavelengths of diffuse media that may (as with mammalian tissue) contain hemoglobin, we can use absorbance parameters, including ratios of absorbance parameters, at two or more wavelengths to derive images representing hemoglobin oxygenation. These raw NIRST mages, or images of blood vasculature and hemoglobin oxygenation may be displayed and recorded 322 directly or may be optionally refined using edges obtained from MRI images from an optional MRI imaging machine 199. MRI imaging typically has far better resolution than imaging, giving image refinement of NIRST images with boundaries derived from MRI imaging an advantage.

To optionally refine raw NIRST images with MRI images, the diffuse media is introduced into 312 the MRI system and MRI images obtained 314. These images are registered 316 to the raw NIRST images. Edges are extracted 318 from the MRI images and used to constrain the voxel-based model while re-executing and refining 320 the fit of optical parameters of the voxel-based model to the recorded light intensity data. The resulting, refined, NIRST images are then displayed 322, and may be displayed in color superimposed on MRI images.

In embodiments, our flexible opto-electronic circuit (sensing) strips with integrated side-firing optical fiber probes use fibers of 200 um core diameter and single-element Si PIN photodiodes (PDs).

Thickness of the strip is about 0.15 mm; in all embodiments it is less than one millimeter thick. The openings for optical fiber probe tip are 3×2 mm windows that enable light from the side firing fiber probes to illuminate the diffuse media. The side firing fiber probes are fed by 200 micrometer core fibers and have 90-degree micro-prisms to direct light onto the diffuse media. To aid co-registration of NIRST images to MRI images, MRI fiducials are installed on each end of each strip, and 3D surface scan markers label the second, fourth, and sixth PDs of each strip. Strips have a thin plastic film coating for cleaning purposes.

### A Summary of Results Achieved

Using a prior MRI NIRST with the breast interface constructed by 16 bulky and heavy fiber bundles, we have imaged more than 100 patients with breast abnormalities and demonstrated optical imaging can enhance the diagnosis power of MRI with or without contrast. In two cases where non-contrast MRI and/or contrast MRI resulted in misdiagnoses when used alone but combined NIRST with MRI enabled correct diagnosis.

Sensitivity, specificity, accuracy, and area under the curve (AUC) of non-contrast MRI when combined with non-contrast MRI NIRST were 94%, 100%, 96%, and 0.95, respectively, whereas they were 94%, 63%, 88%, and 0.81 for contrast MRI alone.

Comparing to Silicon Photomultipliers (SiPMs) and Photomultiplier tubes (PMTs), the single-element Si PIN PDs offers compact size with much better flexibility to fit any tissue shape, similar sensitivity but with a wider spectral range, and lower overall cost. In addition, either PMTs or SiPMs operate under high voltage, and PDs are the only optical sensor that can be used directly on the tissue surface inside of the MR cavity.

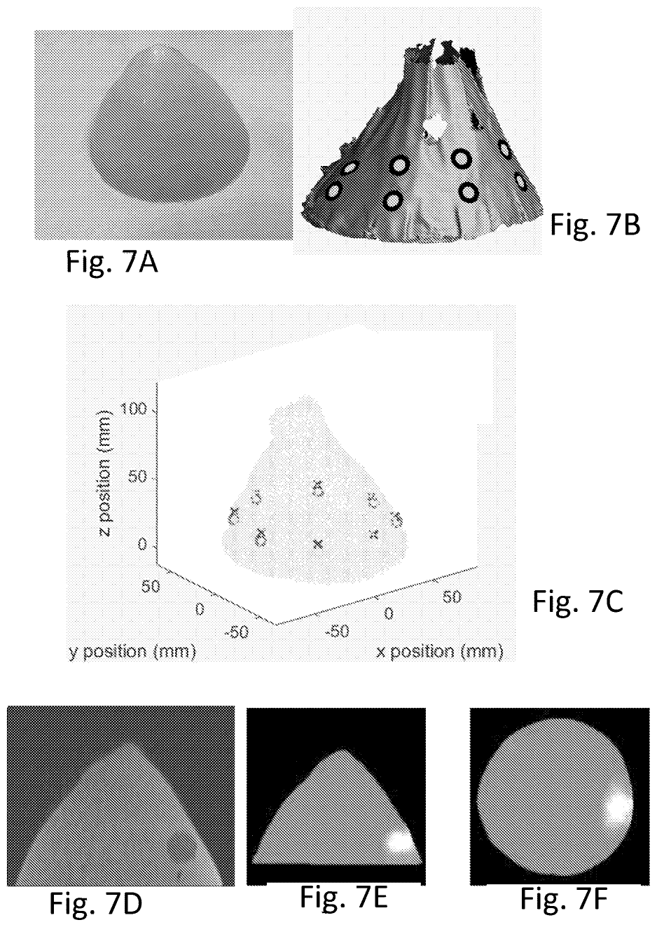

FIG. 7A-7F shows the results from a breast shape phantom with a 15 mm sphere inclusion. A surface scan using a 3D surface camera demonstrated the positions of the strips and PDs (FIG. 7B). According to the 3D scanning images, a volume mesh has been generated and an open-source software, NIRFAST, has been used to calculate the boundary conditions at all nodes marked as sources and detectors and apply to the model to solve the diffusion equation (see FIG. 7C). FIG. 7D is the MR image of the phantom. FIGS. 7E and 7F are the reconstructed 3D images of total hemoglobin concentration from different views. The full volume of the inclusion has been reconstructed (FIG. 7E) and the contrast of inclusion to background is ~1.7, which is very close to its true value of 2 (FIG. 7F).

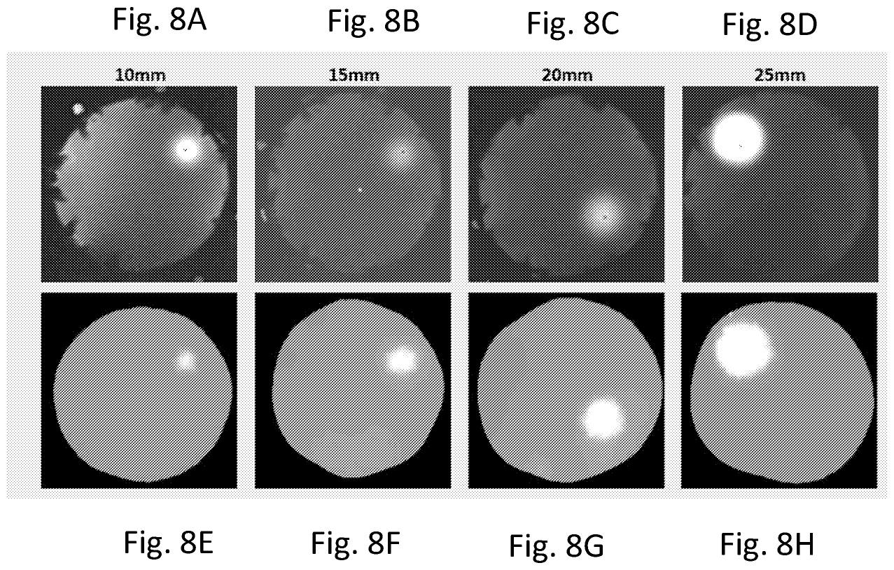

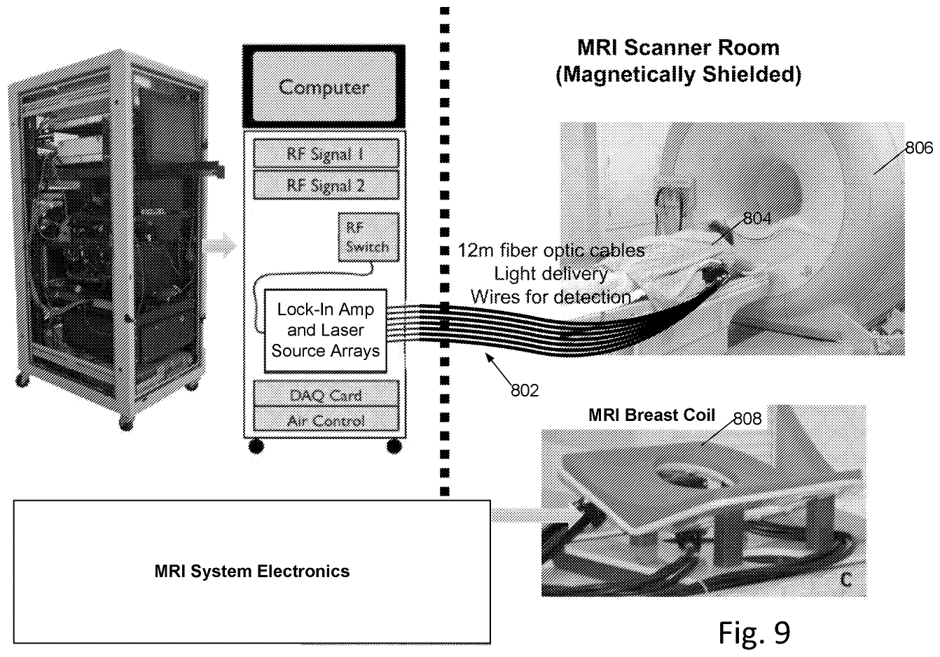

FIG. 8A-8H show the MR and NIRST images of the breast phantoms each with a spherical inclusion to simulate the breast cancer with different sizes in the breast. The diameters of the inclusions are each of 10, 15, 20, and 25 mm respectively. FIGS. 8A, 8B, 8C and 8D illustrate the MR images, while FIGS. 8E, 8F, 8G, and 8H illustrate the corresponding NIRST total hemoglobin images, respectively. Images were obtained using the sensor strips on the breast phantom coupled to an infrared imaging system combined with MRI extracted structure information according to the method of FIG. 9.

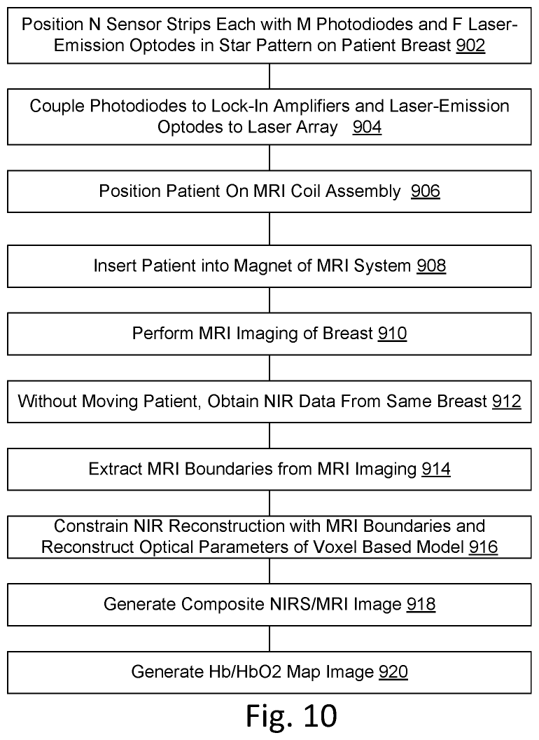

The present sensor strips are formed without ferrous metals and are thus MRI-compatible. With reference to FIGS. 9 and 10, in an embodiment the sensor strips may be coupled together at one end and positioned 902 in a star arrangement like that of FIG. 7B on a diffuse media, in an embodiment the diffuse media is a human patient's breast, in alternative embodiments the diffuse media comprises animal tissue or a phantom. The optical fiber probes of the sensor strips are then coupled 904 through optical fibers 802 through the optical switch 142 (FIG. 1) and near-infrared lasers 146 of strip-associated electronics 144.

Similarly, the photodiodes of the sensor strips are coupled through cables (also 802) to the sense amplifiers and lock-in amplifiers 152. The patient 804 is then positioned 906 on an MRI breast-imaging RF coil 808 and inserted 908 into the bore 806 of an MRI system. MRI imaging is then performed 910.

Without moving the patient, Near infrared imaging spectrographic (NIRS) data is obtained 912 from the same breast or breasts of the patient; in some embodiments the NIRS data is obtained while the MRI imaging is being performed. Then, the volume mesh or regional boundaries are generated 914 from the MRI images and used as constraints while reconstructing 916 optical parameters of a three-dimensional voxel-based model of the breast or breasts using the NIRS data. The optical parameters, including spectrally dependent attenuation, are applied to the MRI boundaries to form 918 a composite NIRST/MRI image useful for diagnostic purposes.

In particular embodiments, the optical parameters are used to map hemoglobin oxygenation and hemoglobin concentration at each voxel of the model, and a three-dimensional image of hemoglobin concentration and oxygen concentration is generated 920. This image of hemoglobin concentration and oxygen concentration is particularly useful in diagnosis since cysts of fibrocystic breast disease tend to be low in hemoglobin and malignant tumors higher in hemoglobin concentration, thus the map of hemoglobin concentration and oxygen concentration gives clues as to tissue types associated with regions in the MRI images.

### Combinations

The features herein described may be combined in various ways. Among combinations of features anticipated by the inventors are:
A near-infrared spectroscopy tomographic (NIRST) imaging strip designated A including multiple photodiodes disposed in a line along a center of a flexible printed circuit, the photodiodes configured to directly receive light from diffuse media the NIRST imaging strip may contact; and at least one, and in some embodiments multiple, optical fiber probes each coupled to an optical fiber and configured to provide light from the optical fibers to the diffuse media.
An NIRST imaging strip designated AA including the NIRST imaging strip designated A where the imaging strip is less than one millimeter thick.
An NIRST imaging strip designated AB including the NIRST imaging strip designated A or AA where each optical fiber probe of the optical fibers directs light around a ninety-degree angle between fiber and the diffuse media.
An NIRST imaging strip designated AC including the NIRST imaging strip designated A, AA, or AB wherein the flexible printed circuit is about 12.4 millimeters wide.
An NIRST imaging strip designated AD including the NIRST imaging strip designated A, AA, AB, or AC wherein the plurality of photodiodes comprises at least two photodiodes and there are at least two optical fiber probes.
An NIRST imaging strip designated AE including the NIRST imaging strip designated A, AA, AB, AC, or AD wherein the plurality of photodiodes comprises six photodiodes and there are at least six optical fiber probes.
An NIRST imaging channel designated AE comprising an NIRST imaging strip including the NIRST imaging strip designated A, AA, AB, AC, AD, or AE and further including at least two selectable wavelength light sources modulated by a waveform generated by a modulation source and coupled through an optical switch to the optical fibers coupled to the optical fiber probes; photodiode sense amplifiers coupled to the photodiodes; and lock-in amplifiers coupled to receive photodiode signals from the sense amplifiers.
An NIRST imaging channel designated AF including the NIRST imaging channel designated AE and further including a data acquisition system coupled between the photodiode sense amplifiers and the lock-in amplifiers.
An NIRST imaging system designate AG including the NIRST imaging channel designated AE or AF and further including and image processor configured to perform NIST imaging using signals from the lock-in amplifiers.
An NIRST imaging system designate AH including the NIRST imaging system designated AG where there are a plurality of NIRST imaging channels.
An NIRST imaging system designated AJ comprising a plurality of the NIRST imaging strips designated A, AA, AB, AC, AD, or AE coupled together in star configuration.
An NIRST imaging system designated AK including the NIRST imaging system designated AJ where the plurality of optical fiber probes are coupled to modulated laser light sources and the photodiodes are coupled through photodiode sense amplifiers to an image processor.
An NIRST imaging system designated AL including the NIRST imaging system designated AJ or AK further including a magnetic resonance imaging (MRI) system.
An NIRST imaging system designated AM including the NIRST imaging system designated AL wherein the image processor is configured to register NIST images to MRI images from the MRI imaging system.
An NIRST imaging system designated AN including the NIRST imaging system designated AM wherein the image processor is configured to extract boundaries from the MRI images and to use those boundaries to improve NIRST images.
An NIRST imaging system designated AO including the NIRST imaging system designated AM where the diffuse media is mammalian tissue.
A method designated B of performing imaging of diffuse media including providing a plurality of NIRST imaging strips each having a row of photodiodes and a slot for at least one optical fiber probe on a flexible circuit board; providing near-infrared light to the diffuse media through a plurality of optical fibers coupled to the optical fiber probes of each NRST imaging strip; receiving light directly from the diffuse media into a plurality of photodiodes of the plurality of NIRST imaging strips; amplifying signals from the plurality of photodiodes; and extracting optical parameters of a voxel-based model of the diffuse media from the amplified signals from the plurality of photodiodes.
A method designated BA including the method designated B where the plurality of NIRST imaging strips are coupled together in star configuration.
A method designated BB including the method designated B or BA further including: obtaining magnetic resonance imaging images of the diffuse media; and constraining the optical parameters of the voxel-based model of the diffuse media with boundaries extracted from the magnetic resonance images while extracting the optical parameters of the voxel-based model of the diffuse media.
A method designated BC including the method designated B, BA, or BB further including: using the extracted optical parameters to map hemoglobin concentration in the diffuse media.
A method designated BD including the method designated B, BA, BB, or BC where the diffuse media is a human breast.
A method designated BE including the method designated B, BA, BB, BC or BD further including using the extracted optical parameters to map hemoglobin oxygenation in the diffuse media.
A method designated BF including the method designated B, BA, BB, BC BD, or BE where each optical fiber probe comprises a 90-degree prism.

Changes may be made in the above methods and systems without departing from the scope hereof. It should thus be noted that the matter contained in the above description or shown in the accompanying drawings should be interpreted as illustrative and not in a limiting sense. Herein, and unless otherwise indicated: (a) the adjective “exemplary” means serving as an example, instance, or illustration, and (b) the phrase “in embodiments” is equivalent to the phrase “in certain embodiments,” and does not refer to all embodiments. The following claims are intended to cover all generic and specific features described herein, as well as all statements of the scope of the present method and system, which, as a matter of language, might be said to fall therebetween.

1. A near-infrared spectroscopy tomographic (NIRST) imaging strip comprising:
a plurality of photodiodes disposed in a line along a flexible printed circuit board;
the plurality of photodiodes configured to receive light from diffuse media when the NIRST imaging strip is contacted with the diffuse media; and
at least one slot within the flexible printed circuit board between the plurality of photodiodes; and
at least one optical fiber probe respectively aligned with the plurality of slots configured to deliver light from at least one light sources to the diffuse media.

2. The NIRST imaging strip of claim 1 where each optical fiber probe directs the light at approximately a ninety-degree angle between probe and the diffuse media.

3. The NIRST imaging strip of claim 1, wherein the imaging strip is less than one millimeter thick and the flexible printed circuit is about 12.4 millimeters wide.

4. The NIRST imaging strip of claim 2, comprising at least two optical fiber probes each aligned with a respective slot.

5. The NIRST imaging strip of claim 4 wherein the plurality of photodiodes comprises six photodiodes and there are at least six optical fiber probes.

6. An NIRST imaging channel comprising an NIRST imaging strip of claim 1 and further comprising:
at least two selectable wavelength light sources modulated by a waveform generated by a modulation source and coupled through an optical switch through optical fibers to the optical fiber probes;
photodiode sense amplifiers coupled to the photodiodes; and
lock-in amplifiers coupled to receive photodiode signals from the sense amplifiers.

7. The NIRST imaging channel of claim 6 further comprising a data acquisition system coupled between the photodiode sense amplifiers and the lock-in amplifiers.

8. An NIRST imaging system comprising at least one NIRST imaging Strip of claim 6 and an image processor configured to perform NIRST imaging using signals from the lock-in amplifiers.

9. An NIRST imaging system of claim 8 comprising a plurality of NIRST imaging channels.

10. An NIRST imaging system comprising a plurality of the NIRST imaging strips of claim 1 coupled together in star configuration.

11. The NIRST imaging system of claim 10 the at least one optical fiber probe comprising a plurality of optical fiber probes coupled to modulated laser light sources and the photodiodes being coupled through photodiode sense amplifiers to an image processor.

12. An NIRST imaging system of claim 11 further comprising a magnetic resonance imaging (MRI) system.

13. An NIRST imaging system of claim 12 wherein the image processor is configured to register NIRST images to MRI images from the MRI imaging system.

14. An NIRST imaging system of claim 13 wherein the image processor is configured to extract boundaries from the MRI images and to use those boundaries to improve NIRST images.

15. An NIRST imaging system of claim 14 where the diffuse media is mammalian tissue.

16. A method of performing imaging of diffuse media comprising:
providing a plurality of NIRST imaging strips each having a row of photodiodes and a slot for at least one optical fiber probe on a flexible circuit board;
providing near-infrared light to the diffuse media through a plurality of optical fibers coupled to the optical fiber probes of each NRST imaging strip;
receiving light directly from the diffuse media into a plurality of photodiodes of the plurality of NIRST imaging strips;
amplifying signals from the plurality of photodiodes; and
extracting optical parameters of a voxel-based model of the diffuse media from the amplified signals from the plurality of photodiodes.

17. The method of claim 16 where the plurality of NIRST imaging strips are coupled together in star configuration.

18. The method of claim 16 further comprising:
obtaining magnetic resonance imaging images of the diffuse media; and
constraining the optical parameters of the voxel-based model of the diffuse media with boundaries extracted from the magnetic resonance images while extracting the optical parameters of the voxel-based model of the diffuse media.

19. The method of claim 18 further comprising using the extracted optical parameters to map hemoglobin concentration in the diffuse media.

20. The method of claim 19 where the diffuse media is a human breast.

21. The method of claim 19 further comprising using the extracted optical parameters to map hemoglobin oxygenation in the diffuse media.

22. The method of claim 16, each optical fiber probe comprising a 90-degree prism.
